(19) United States
(12) Patent Application Publication
(10) Pub. No.: US 2023/0377681 A1
(43) Pub. Date: Nov. 23, 2023

(54) SYSTEM AND METHOD FOR IDENTIFYING THERAPEUTICS FOR A GIVEN ILLNESS USING MACHINE LEARNING

(71) Applicants: Supratik MUKHOPADHYAY, Baton Rouge, LA (US); Adam BESS, Baton Rouge, LA (US); Michal BRYLINSKI, Baton Rouge, LA (US)

(72) Inventors: Supratik MUKHOPADHYAY, Baton Rouge, LA (US); Adam BESS, Baton Rouge, LA (US); Michal BRYLINSKI, Baton Rouge, LA (US)

(73) Assignee: Board of Supervisors of Louisiana State University and Agricultural and Mechanical College, Baton Rouge, LA (US)

(21) Appl. No.: 18/030,258

(22) PCT Filed: Oct. 6, 2021
(86) PCT No.: PCT/US21/71750

§ 371 Date: Apr. 4, 2023

Related U.S. Application Data
(60) Provisional application No. 63/088,301, filed on Oct. 6, 2020.

(51) Int. Cl. G16B 15/30; G16B 40/30

(52) U.S. Cl. CPC G16B 15/30; G16B 40/30

(57) ABSTRACT

Systems, methods, and non-transitory computer-readable storage media for identifying potential therapeutics for pathogens (bacterial or viral). The system can receive a plurality of molecules known to have interactions with a pathogen, fragment the plurality of molecules into a plurality of linkers and a plurality of rigids, and remove redundancies from the plurality of linkers and the plurality of rigids. The system can then identify a plurality of possible molecules formed from the fragments, evaluate those possible molecules for toxicity, and analyze the non-toxic candidates using a convolutional neural network associated with the pathogen. The final candidates can then be used for in-vivo testing.

### CROSS REFERENCE

The present disclosure claims priority to U.S. Provisional Patent Application 63/088,301, filed Oct. 6, 2020, the contents of which are incorporated herein in their entirety.

### BACKGROUND

### 1. Technical Field

The present disclosure relates to identifying potential new therapeutics for treating pathogens, and more specifically to using a combination of filters, machine learning/Artificial Intelligence (A.I.), and graph convolutional networks to rank potential candidate drugs for treating bacterial and viral pathogens.

### 2. Introduction

Graph Convolutional Networks (GCNs) allows extremely computationally intensive graph data to be reduced to a form that allows the data to be processed, without losing features which are important for obtaining accurate predictions regarding the input data. This is generally accomplished by reducing the dimensionality (also called “flattening”) of the input data to a point where the computational power required to process the data is likewise reduced, allowing for classification or other determinations regarding the input data to be made.

With respect to therapeutics, GCNs could allow researchers to speed up the time required to identify potential new drugs or chemical combinations which could be used to mitigate the effects of various pathogens, such as bacterial or viral infections.

### SUMMARY

Additional features and advantages of the disclosure will be set forth in the description which follows, and in part will be obvious from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are systems, methods, and non-transitory computer-readable storage media providing a technical solution to the technical problem described. A method for identifying therapeutics for a given illness, as described herein, can include: obtaining, at a processor, a plurality of building block substructures contained within a plurality of candidate drugs for the given illness; executing, via the processor, an artificial intelligence algorithm which combines one or more of the plurality of building block substructures according to predefined rules to generate molecules are formed in a chemically sound manner, resulting in first candidate molecules; filtering, via the processor, the first candidate molecules for toxicity and ease of manufacture, resulting in second candidate molecules; generating, via the processor, a mathematical representation of physical contacts between proteins in a host cell; receiving a dataset of drug-target interactions comprising drug-protein interactions for the pathogen; generating, via the processor using the mathematical representation, a graph convolutional network comprising a plurality of nodes and a plurality of edges connecting the plurality of nodes, by: organizing the proteins in the host cell, proteins identified within the pathogen, and building block substructures within the second candidate molecules into neighborhoods of nodes according to protein structure and protein source, where each protein is associated with a node in the plurality of nodes, and each node is summarized by a feature matrix; and connecting the plurality of nodes with the plurality of edges based at least in part on one interaction selected from protein-protein interactions within the host cell, protein-protein interactions within the pathogen, and interactions between building block substructures and pathogen proteins, where each edge in the plurality of edges comprises features describing the interaction, resulting in the graph convolutional network; ranking interactions with the pathogen based on the features of edges in the plurality of edges which connect to proteins associated with the pathogen, resulting in ranked interactions; and generating a list of final candidate drugs based on the ranked interactions.

An example system configured as disclosed herein can include: a processor; and a non-transitory computer-readable storage medium having stored therein instructions which, when executed by the processor, cause the processor to perform operations comprising: receiving a plurality of molecules known to have interactions with a pathogen; fragmenting the plurality of molecules into a plurality of linkers and a plurality of rigids; removing redundancies from the plurality of linkers and the plurality of rigids, resulting in fragments; identifying a plurality of possible molecules formed from the fragments; evaluating the plurality of possible molecules for toxicity, resulting in non-toxic possible candidates; analyzing the non-toxic possible candidates using a convolutional neural network associated with the pathogen, resulting in final candidates; and outputting the final candidates for in-vivo testing.

An example non-transitory computer-readable storage medium configured as disclosed herein can have stored instructions which, when executed by a processor, cause the processor to perform operations including: receiving a plurality of molecules known to have interactions with a pathogen; fragmenting the plurality of molecules into a plurality of linkers and a plurality of rigids; removing redundancies from the plurality of linkers and the plurality of rigids, resulting in fragments; identifying a plurality of possible molecules formed from the fragments; evaluating the plurality of possible molecules for toxicity, resulting in non-toxic possible candidates; analyzing the non-toxic possible candidates using a convolutional neural network associated with the pathogen, resulting in final candidates; and outputting the final candidates for in-vivo testing.

### BRIEF DESCRIPTION OF THE DRAWINGS

FIG. 1 illustrates a first example method according to the invention;

FIG. 2 illustrates a first example of fragmentation of molecules;

FIG. 3 illustrates a second example of fragmentation of molecules and synthesis of a bioactive;

FIG. 4A illustrates a first graph representation of rigids and linkers;

FIG. 4B illustrates a second graph representation of rigids and linkers;

FIG. 4C illustrates a third graph representation of rigids and linkers;

FIG. 4D illustrates a fourth graph representation of rigids and linkers;

FIG. 4E illustrates a fifth graph representation of rigids and linkers;

FIG. 5 illustrates an example of molecular synthesis;

FIG. 6 illustrates an example of fragmentation and molecular synthesis;

FIG. 7 illustrates an example of using machine learning to develop a toxicity score;

FIG. 8 illustrates an example of a graph convolutional network;

FIG. 9 illustrates an example method embodiment; and

FIG. 10 illustrates an example computer system.

### DETAILED DESCRIPTION

Various embodiments of the disclosure are described in detail below. While specific implementations are described, it should be understood that this is done for illustration purposes only. Other components and configurations may be used without parting from the spirit and scope of the disclosure.

Exemplary technical problems associated with the use of GCNs in such endeavors include the amount of data being input into the graph to form a GCN is too large, which results in too many potential candidate drugs to be meaningfully reviewed by scientists, and the data format of the constructed GCN.

One exemplary, non-limiting, practical application to the technical problem noted above is to fragment known portions of previously analyzed molecules, analyze and filter potential combinations from those fragments based on toxicity, and evaluate the resulting potential combinations using a Graph Convolutional Network (GCN). From the GCN evaluation, a ranking of the final candidates/potential new molecules can be provided to researchers for additional tests against the given pathogen.

The particular order of fragmenting, building new molecules from those fragments, testing the new molecules for toxicity, and then providing those molecules which clear the toxicity test to a processor for testing as part of a graph convolutional network test represent a preliminary culling of the number of molecules which a system needs to evaluate, allows for an overall faster computation by a computer system of which molecules have a highest likelihood of effectiveness. In addition, the particular way in which the convolutional network is generated and used to evaluate the effectiveness of potential molecules represents an improvement in evaluating those molecules which have already been filtered.

Compounds showing promising activity identified by high throughput screening as initial hits are filtered and modified to generate lead compounds, which satisfy basic drug-likeliness properties. These lead compounds are further optimized to enhance the potency toward the target protein as well as to reduce their non-selectivity and toxicity.

In order to enhance the chemical diversity of virtual screening libraries, large collections of drug-like compounds can be generated through combinatorial chemistry. Since constructing and screening the entire chemical space is not feasible even with the most advanced computers, building extensive yet targeted libraries is critical for the success of virtual screening. A number of fragment- and atom-based techniques have been developed to generate novel chemical compounds for virtual screening, including binding-site point connection methods (LUDI), fragment connection methods (LEA3D, LigBuilder, and eSynth), sequential build-up algorithms (LEGEND and SPROUT), and random connection techniques (CoG and Flux). These de novo methods can require an initial set of building blocks or molecular fragments, which ultimately control the properties of the resulting screening compounds and their affinity toward the target protein. Consequently, there is a great interest in efficient fragmentation techniques to generate sets of chemically feasible building blocks for the subsequent molecular synthesis. Retrosynthetic combinatorial analysis procedure (RECAP) and breaking retro-synthetically interesting chemical substructures (BRICS) are examples of systematic fragmentation methods. In RECAP, compounds are dissected based on a set of 11 bond types, following simple rules such as leaving cyclic bonds and alkyl groups smaller than five carbons intact. These rules ensure that major structural features of organic compounds, such as ring motifs, are preserved. BRICS expands the bond type criteria used by RECAP from 11 to 16, taking into account the chemical environment of each bond type and the surrounding substructures. Additional filters can also be applied in order to prevent generating small and unwanted fragments. Other configurations can extract and classify chemical scaffolds by pruning side chains and removing peripheral ring moieties.

In general, the performance of fragment-based chemical synthesis tools such as eSynth, CONFIRM, and AutoGrow can be improved by employing building blocks annotated with empirical connectivity patterns. Although this information could help explore pharmacologically relevant regions of the diverse chemical space, many existing fragmentation tools, e.g. Fragmenter and molBLOCKS, do not consider the chemical context of the fragments. In other words, the connectivity information on a fragment is not stored while extracting building blocks. To address this issue, the systems and methods disclosed decompose either a single ligand or a library of compounds into two types of chemical building blocks, rigids and the connecting linkers. The resulting complete and nonredundant sets of building blocks can be annotated with the comprehensive connectivity information in order to facilitate the construction of novel compounds with combinatorial synthesis software. Moreover, this process can be parallelized to decrease the computing time required to analyze large collections of molecules.

The fragmentation process, as disclosed herein, can employ a graph-based notation, where molecules are sets of nodes representing atoms connected by edges corresponding to chemical bonds. A fragment is a substructure, which has either all or only some atoms and bonds of a given molecule; fragments are categorized as either rigids or linkers. Given a collection of molecules, the complete set of unique fragments is constructed in two steps. The first step involves creating an initial set of fragments, whereas the second step guarantees the uniqueness of the resulting set of fragments.

Here a set of molecules are first decomposed into constituent fragments with the BRICS algorithm, implemented in RDKit. Chemical compounds are broken down into larger moieties called rigids connected by linkers based on 16 chemical environments defined by the BRICS model

Algorithm S1. Molecular fragmentation.
Algorithm S1

| | |
|---|---|
| 1: | procedure Fragment(Set<Molecule> M) |
| 2: | List<Brick> B := ∅ |
| 3: | List<Linker> L := ∅ |
| 4: | for each m ∈ M |
| 5: | for each f ∈ FragmentOnBRICSBonds(m) |
| 6: | if f.isBrick( ) then |
| 7: | l := f.RemoveDummyAtoms( ) |
| 8: | f.RemoveHydrogen( ) |
| 9: | f.AddAppendix(l) |
| 10: | Bₘ := Bₘ ∪ [f] |
| 11: | end if |
| 12: | end for |
| 13: | B := B ∪ Bₘ |
| 14: | L := L ∪ ComputeLinkers(m, Bₘ) |
| 15: | end for |
| 16: | return [symbol] B, L [symbol] |
| 17: | end procedure |

A brick fragment is a molecular construct having at least four non-hydrogen atoms. Subsequently, rigids are removed from a molecule and the remaining fragments are classified as linkers

Algorithm S2. Linker extraction.
Algorithm S2

| | |
|---|---|
| 1: | procedure ComputeLinkers(Molecule m, List<Brick> Bₘ) |
| 2: | for each b ∈ Bₘ |
| 3: | m.removeBrick(b) |
| 4: | end for |
| 5: | List<Linker> [symbol] := m.RemainingFragments( ) |
| 6: | for each l ∈ [symbol] |
| 7: | l.AddAppendix(m) |
| 8: | end for |
| 9: | return [symbol] |
| 10: | end procedure |

Broken bonds are replaced by dummy atoms, which are placeholders for those atoms removed from a particular bond. The complete information, including the type of atoms involved in those bonds that were broken, is stored for each brick in order to provide empirical connectivity patterns. Linkers have different auxiliary connectivity information, i.e. these fragments are annotated only with the maximum number of bonds at various positions.

First is a procedure for the automatic identification and extraction of molecular fragments from chemical compounds. The extraction process utilizes the PDBQT file format containing a central rigid fragment, labeled as ROOT, from which zero or more rotatable bonds protrude. The sets of atoms connected through rotatable bonds are organized as BRANCHes, and at the beginning and end of each BRANCH section, the serial numbers of the two atoms forming a rotatable bond are recorded. First, the system identifies all rigid moieties, where a rigid fragment is defined as a set of at least four non-hydrogen atoms connected by non-rotatable bonds. The remaining parts are extracted as flexible linkers. If two linker fragments are attached to each other, these will be connected to form a longer linker. Failing to construct longer linkers from shorter fragments would limit the library to contain only very short linkers. Furthermore, the system tracks the connectivity between individual fragments, so that chemically feasible compounds can be synthesized using a graph-based algorithm. Every fragment is stored in the Structure Data Format (SDF) (examples of which follow) containing the 3D coordinates of all atoms and the corresponding atomic types as well as the connectivity information. The following SYBYL chemical types are used for ligand atoms: carbon (C.1, C.2, C.3, C.ar and C. cat), nitrogen (N.1, N.2, N.3, N.4, N.am, N.ar and N.p13), oxygen (O.2, O.3 and O.co2), phosphorous (P.3), sulfur (S.2, S.3, S.O and S.O2), and halogens (Br, Cl, F, I). (Atomic designations following the Cambridge Structural database, available at https://www.sdsc.edu/CCMS/Packages/cambridge/pluto/atom_types.html).

Example SDF file I with component explanation

[symbol]

| | |
|---|---|
| [symbol] | [symbol] |

[symbol]

[symbol]

[symbol]

[symbol]

[symbol]

[symbol]

[symbol]

[symbol]

[symbol]

[symbol]

[symbol]

| | | | | | | | | | | | | | | | |
|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

```
1 [symbol]  1 [symbol]
1 [symbol]  1 [symbol]
2 [symbol]  1 [symbol]
2 [symbol]  1 [symbol]
3 [symbol]  1 [symbol]
4 [symbol]  1 [symbol] [symbol]
END
> <ATOMTYPES>

[symbol]

[symbol]

[symbol]

[symbol]

[symbol]

[symbol]






> < [symbol]  >

[symbol]

> <fragments similar>
/ [symbol]  /output/ [symbol] /b-

[symbol]

[symbol]

The auxiliary information included in brick SDF files:
  <ATOMTYPES> Atom types according to SYBL ordered according to the atom section
  containing Cartesian coordinates.
  <BRANCH [symbol] atom-number eligible-atomtype- [symbol] -connect> List of all possible bonds
  for this brick. The 1^{st} column is the atom index followed by atom types allowed to be
  connected at this position. For example, the 6^{th} atom in the brick fragment shown above,
  which is [symbol]  can connect to a [symbol]  atom.
  <fragments similar> After removing redundancy, only one construct is kept for each
  unique fragment. This section tracks back all similar fragments that have been consolidated. [symbol]
indicates data missing or illegible when filed
```

Example SDF file II with component explanation

[symbol]

| | |
|---|---|
| [symbol] | [symbol] |

[symbol]

[symbol]

[symbol]

[symbol]

[symbol]

[symbol]

[symbol]

[symbol]

[symbol]

[symbol]

[symbol]

| | | | | | | | | | | | | | | | |
|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

```
1 3 2 0 [symbol]
3 1 0 [symbol]
END
> <MAX-NUMBER-OF-CONTACTS ATOMTYPES>

[symbol]

[symbol]

[symbol]

[symbol]




The auxiliary information included in linker SDF files:
  < MAX-NUMBER-OF-CONTACTS ATOMTYPES> The 1^{st} column shows the maximum number
  of connections allowed at every atom, which are ordered according to the atom section
  containing Cartesian coordinates. Atom types are listed in the 2^{nd} column. For example, the
  second line in this section in the linker fragment shown above ( [symbol] ) means that the 2^{nd}
  atom is [symbol]  and it can form only one connection to other atoms. [symbol]
indicates data missing or illegible when filed
```

Because one objective of an effective fragmentation procedure is to employ the resulting fragments in a synthesis procedure, the cardinality of the final set of fragments is critical. On that account, the disclosed methods attempt to minimize the size of sets of rigids and linkers by removing redundancy with a partitioning and sieve-based removal scheme. Redundancy can be removed from molecular fragments extracted from multiple compounds by consolidating the connectivity information and deleting identical moieties. For instance, if different parent molecules have a similar fragment, it suffices to have only one file for this moiety. However, if this fragment is connected to different atom types in distinct parent molecules, all possibilities should be retained. Therefore, this information is deposited as one copy of the fragment representing all possible connections, i.e., this information is consolidated to create only one rigid moiety with two possible connections.

Algorithm S3. Removal of redundant fragments.
Algorithm S3

| | |
|---|---|
| 1: | procedure RemoveRedundancy(List<Fragment> F) |
| 2: | List<Fragment> U := ∅ // Unique fragment set |
| 3: | List<Set<Fragment>> [symbol] /~ := Partition(F) |
| 4: | for each P ∈ [symbol] /~ |
| 5: | while P ≠ ∅ |
| 6: | f₀ = P.removeFirst( ) |
| 7: | U := U ∪ [f₀] |
| 8: | for each f ∈ P |
| 9: | if f₀ = f then P := P \ [symbol] f [symbol] |
| 10: | end for |
| 11: | end while |
| 12: | end for |
| 13: | return U |
| 14: | end procedure |
| 15: | procedure RemoveRedundancy(List<Brick> [symbol] , List<Linker> L) |
| 16: | [symbol] := RemoveRedundancy( [symbol] ) |
| 17: | L := RemoveRedundancy(L) |
| 18: | end procedure |

[symbol]

indicates data missing or illegible when filed

Two fragments are equivalent if the Tanimoto coefficient (TC) calculated for topologically constrained maximum common substructures by the kcombu (K(ch)emical structure COMparison using the BUild-up algorithm) program is equal to 1.0. Information on equivalent atoms provided by kcombu as well as their connectivity information is then used to consolidate identical fragments into a single, unique construct.

The system can then identify potential synthetic molecules by computationally synthesizing molecules for virtual libraries. To summarize, an exhaustive graph-based search algorithm can be used to reconnect chemical building blocks procured from bioactive compounds following realistic connectivity patterns. Rather than focusing on a certain scaffold, the moieties used for synthesis can come from active ligands of a specific target protein. Thus, the resulting chemical space can be highly diverse, yet targeted. Given a set of initial molecules, this method can generate new compounds to populate the pharmacologically relevant space. The protocols mimic a real application, where one expects to discover novel compounds based on a small set of already developed bioactive compounds. Equally important, the method allows adding active subunits to an existing compound in order to generate a large library of prototypes of the modified ligand. Such libraries can be examined by molecular docking to explore those modifications yielding the highest binding affinity to the protein target.

A rigid fragment carries connectivity information indicating those atoms from which a rigid fragment was originally branched and the corresponding atom types to which it was connected. On the other hand, linkers contain information only on the number of allowed contacts at every atom, which is sufficient to create bonds with rigid fragments (linkers cannot bind to each other). The number of connections in a linker cannot exceed the maximum number of covalent bonds. Thus, a linker is saturated with hydrogen atoms and the maximum number of bonds allowed for each atom in the linker file can be reported. Noticeably, long linkers with the extensive connectivity pose a risk of expanding the molecular search space to an unmanageable size. Therefore, unsaturated linkers can also be built to store only the number of original connections, regardless of the maximum capacity of their atoms to create covalent bonds. In contrast to saturated linkers, using unsaturated linkers with substantially less connectivity considerably restricts the search space.

The disclosed method considers molecular bonding over a given set of rigid and linker fragments restricted by the laws of chemistry. Molecular synthesis is a fixed-point approach to generate a complete set of molecules given a set of fragments. A fragment-based approach to synthesis can result in an infinite molecular search space unless an upper bound for molecular size is specified. Even with reasonable upper bounds imposed on the molecule size, the synthesis process may result in 10⁸ molecules or more. It is therefore highly desirable to develop an efficient algorithm for molecular synthesis that is complete, i.e. all possible molecules that can be synthesized under chemical and physical constraints are guaranteed to be generated. For expository purposes, a k-molecule is referred to as a molecule that is composed of k molecular fragments. Algorithm 4 uses a level-based approach to molecular synthesis, where all molecules in a level are composed of the same number of fragments.

Algorithm 4

| | | |
|---|---|---|
| 1: | procedure Synthesize(L, [symbol] , MAX) | // linkers L, rigids [symbol] , MAX |
| 2: | Set<Molecule> M[MAX]; | // 2D array of unique molecules |

| | |
|---|---|
| 3: | [symbol] := M[1] := L ∪ [symbol] |
| 4: | for [symbol] := 2 to MAX |
| 5: | for each f ∈ [symbol] |
| 6: | for each m ∈ M[ [symbol] − 1] |
| 7: | M[ [symbol] ].AddAll(Compose(f,m)) |
| 8: | end for |
| 9: | end for |
| 10: | end for |
| 11: | return [symbol] M[ [symbol] ] |
| 12: | end procedure |

[symbol]

indicates data missing or illegible when filed

In Algorithm 4, line 3 initializes the synthesis process by storing 1-molecules (i.e. fragments) in the array M (at index 1). On lines 4 to 10, the system exhaustively synthesizes each new level from 2- to MAX-molecules, where MAX is an upper bound parameter set by the user. The system stores all k-molecules at index k in M. The synthesis process is performed by the Compose(m1,m2) function which takes two molecules m1 and m2 and combines them together in all possible orientations as dictated by allowable bonding vertices (connectivity information) in the graph representation of each molecule. Compose returns a set of molecules that meet the stated constraints, including Lipinski compliance, to be added to the appropriate set of k-molecules. Last, on line 11, the system combines the sets of all synthesized molecules into a single collection that is returned.

The level-based approach to molecular synthesis used in Algorithm 4 is malleable depending on computational constraints. For example, Algorithm 4 implies that the synthesis of level k must complete prior to level k$+$1 starting. However, an astute observer will recognize that Algorithm 4 can easily be modified for a multi-threaded approach in which level k is a producer for level k$+$1, the consumer. Thus, if each level maintains a thread acting as producer and consumer, the synthesis process can be expedited.

Algorithm 5

| 1: | procedure Synthesize(L, [symbol] , MAX) | // linkers L, rigids [symbol] , MAX |
|---|---|---|
| 2: | Set<Molecule> M[MAX] := {∅} | // array of lists of molecules |
| 3: | Set<Molecule> W[MAX]:= {∅} | // init worklists to empty |
| 4: | W[1] := [symbol] := M(1) := L ∪ [symbol] | // fragments are level 1 |
| 5: | while [symbol] W( [symbol] ).empty( ) | // empty W[ [symbol] = 1] ⇒ [symbol] ≥ 2 complete |
| 6: | SynthesizeHelper(l, W, [symbol] , MAX, M) | |
| 7: | end while | |
| 8: | return [symbol] M[ [symbol] ] | |
| 9: | end procedure | |
| | // SynthesizeHelper: inductively processes molecules from | |
| | // level [symbol] to [symbol] + 1 adhering to capacities at each level | |
| 10: | procedure SynthesizeHelper( [symbol] , W, [symbol] , MAX, M) | |
| 11: | while !W[ [symbol] ].empty( ) | |
| | // check [symbol] + 1 capacities; process [symbol] -molecules, if any | |
| 12: | while [symbol] W[ [symbol] + 1].atCapacity( ) and [symbol] W[ [symbol] ].empty( ) | |
| 13: | m := W[ [symbol] ].pop( ) | |
| 14: | M[ [symbol] ].Add(m) | |
| 15: | for each f ∈ [symbol] F | // compose from [symbol] into [symbol] + 1 |

| 16: | W[ [symbol] + 1].AddAll(Compose(f, m) [symbol] | |
|---|---|---|
| 17: | end for | |
| 18: | end while | |
| 19: | if ( [symbol] ≤ MAX) | // adhere to upper bound synthesis level |

| | | |
|---|---|---|
| 20: | SynthesizeHelper ( [symbol] + 1, W, [symbol] , MAX, M) | // process [symbol] + 1 |

| | |
|---|---|
| 21: | end if |
| 22: | end while |
| 23: | end procedure |

[symbol]

indicates data missing or illegible when filed

There may also be a bounded alternative of Algorithm 4. In Algorithm 5 (the bounded, level-based molecular synthesis alternative), the system maintains an array of worklists (line 3), one for each level that has an explicit capacity. If the system reaches the capacity of a worklist at level, the system forgoes processing the remaining items at level % and inductively complete processing of all molecules at level [symbol] $+$1 (Line 12). Otherwise, from lines 15 to 17 the system composes a molecule from level [symbol] with all of the fragments in F into level [symbol] $+$1 molecules as before. We note that the approach in Algorithm 5 is appropriate for either serial or parallel syntheses depending on the availability of computational resources.

Molecular synthesis is limited by physical restrictions on molecules, but more so limited by time and space. An efficient synthesis must overcome time and space considerations, generate molecules within the physical restrictions, but do so without redundancy. Using either Algorithm 4 or Algorithm 5 results in a significant redundancy present in synthesized molecules. A typical synthesis scenario from a basis of fragments will generate hundreds of millions of molecules, which makes storing these molecules in memory infeasible. Therefore, eliminating molecular redundancy requires a memoryless technique, such as a series of Bloom filters.

A Bloom filter is a probabilistic data structure that is efficient in terms of time and space. The main purpose of a Bloom filter is to determine whether an element is in a given set. Let M be a set of molecules and M a molecule. A Bloom filter is guaranteed to answer the query M∉M if molecule M is an element in set M. Since a Bloom filter is a probabilistic data structure, it is subject to false positives, i.e. a query may return M∉M when in fact M is not in M, however,

the rate of false positives can be controlled. A Bloom filter is based on the number of bits in the filter array b, the number of distinct hash functions h, and the number of elements n expected to be inserted into the filter. Assuming all hash functions respectively hash input elements uniformly to all b bits in the target array, the rate of false positives for an element M∈M is given by P(M∈M)=(1−e^{-nh/b})^{h}. It can be shown that to minimize the rate of false positives, the required number of hash functions h is given by

$$h = \frac{b \times ln 2}{n} .$$

If p is the desired false positive rate, it can also be shown that the required number of bits is

$$b = \frac{n \times ln p}{{( ln 2 )}^{2}} .$$

Consider a molecular Bloom filter F in which the system tolerates a 1% false positive rate for 10⁸ molecules. In this case, the system requires b=9.585×10⁸≈120 megabytes with h=7 hash functions. This means each addition of a molecule to F and each query on F is subject to the worst case of O(h)=7 hashings.

Molecular synthesis requires a string representation of molecules. In particular, a molecule M is represented using the Simplified Molecular-Input Line-Entry System (SMILES) specification in the Bloom filter. The Compose function in Algorithm 5 can be modified to include several Bloom filters, such as a single, overall filter F and a filter [symbol] for each level. When a level [symbol] molecule M is synthesized, the system first checks whether M has been previously synthesized by querying [symbol] . If the molecule has not been synthesized (M∈ [symbol] ), the system adds M to [symbol] and queries F. If M∈F, the system adds M to F and proceeds as in Algorithm 5 by adding M to the level [symbol] queue to be processed into level ( [symbol] +1) molecules. The global Bloom filter F requires the most memory, but ensures that molecules containing different number of fragments with the same SMILES representation are filtered as redundant.

The architecture of the synthesis described herein reflects a simple input/output paradigm with a black-box synthesizer. The input is a set of rigid and linker fragments in SDF format. Each SDF file is parsed using some functionality of Open Babel (or similar software) into a graph-based representation of the corresponding rigids and linkers. From the set of linkers and rigids, the Synthesizer implements Algorithm 5 to construct new compounds.

The system can also predict the synthetic accessibility and the toxicity of molecules. In contrast to other approaches for predicting toxicity by employing manually-crafted descriptors, the system described herein can implement a generic model to estimate the toxicity directly from the molecular fingerprints of chemical compounds. Consequently, it may be more effective against highly diverse and heterogeneous datasets. Machine learning models are trained and cross-validated against a number of datasets comprising known drugs, potentially hazardous chemicals, natural products, and synthetic bioactive compounds. The system also conducts a comprehensive analysis of the chemical composition of toxic and non-toxic substances. Overall, the disclosed system effectively estimates the synthetic accessibility and the toxicity of small organic compounds directly from their molecular fingerprints. As the primary application, this technique can be incorporated into high-throughput pipelines constructing custom libraries for virtual screening to eliminate from CADD (Computer Aided Drug Design) those drug candidates that are potentially toxic or would be difficult to synthesize.

Numerous machine learning-based techniques have been developed to reveal complex relations between chemical entities and their biological targets. A first algorithm used in the learning process is the Restricted Boltzmann Machine (RBM), an undirected graphical model with a visible input layer and a hidden layer. In contrast to the unrestricted Boltzmann Machine, in which all nodes are connected to one another, all inter-layer units in the RBM are fully connected, while there are no intra-layer connections. The RBM is an energy-based model capturing dependencies between variables by assigning an “energy” value to each configuration. The RBM is trained by balancing the probability of various regions of the state space, viz. the energy of those regions with a high probability is reduced, with the simultaneous increase in the energy of low-probability regions. The training process involves the optimization of the weight vector through Gibbs sampling.

The Deep Belief Network (DBN) is a generative probabilistic model built on multiple RBM units stacked against each other, where the hidden layer of an unsupervised RBM serves as the visible layer for the next sub-network. This architecture allows for a fast, layer-by-layer training, during which the contrastive divergence algorithm is employed to learn a layer of features from the visible units starting from the lowest visible layer. Subsequently, the activations of previously trained features are treated as a visible unit to learn the abstractions of features in the successive hidden layer. The whole DBN is trained when the learning procedure for the final hidden layer is completed. It is noteworthy that DBNs are first effective deep learning algorithms capable of extracting a deep hierarchical representation of the training data.

An exemplary system can utilize a DBN implemented in Python with Theano and CUDA to support Graphics Processing Units (GPUs). The SAscore (Synthetic Accessibility: The predicted value of the ease of synthesis/manufacture in a wetlab/factory for a given molecule) is predicted with a DBN architecture consisting (as an example) of a visible layer corresponding to a 1024-bit Daylight fingerprint and three hidden layers having 512, 128, and 32 nodes. The L2 regularization can be employed to reduce the risk of overfitting. The DBN can employ an adaptive learning rate decay with an initial learning rate, a decay rate, mini-batch size, the number of pre-training epochs, and the number of fine-tuning epochs of 0.01, 0.0001, 100, 20, and 1000, respectively.

Finally, the Extremely Randomized Trees, or Extra Trees (ET), algorithm can be used to predict the toxicity of drug candidates. A simpler algorithm (compared to the ET algorithm) can also be used because classification is generally less complex than regression. Classical random decision forests construct an ensemble of unpruned decision trees predicting the value of a target variable based on several input variables. Briefly, a tree is trained by recursively partitioning the source set into subsets based on an attribute value test. The dataset fits well the decision tree model because each feature takes a binary value. The recursion is completed when either the subset at a node has an invariant target value or when the Gini impurity reaches a certain threshold. The output class from a decision forest is simply the mode of the classes of the individual trees. The ET classifier is constructed by adding a randomized top-down splitting procedure in the tree learner. In contrast to other tree-based methods commonly employing a bootstrap replica technique, ET splits nodes by randomly choosing both attributes and cut-points, as well as it uses the whole learning sample to grow the trees. Random decision forests, including ET, are generally devoid of problems caused by overfitting to the training set because the ensemble of trees reduces model complexity leading to a classifier with a low variance. In addition, with a proper parameter tuning, the randomization procedure in ET can help achieve robust performance even for small training datasets.

Various compound datasets can be used to evaluate toxicity. The first two example sets, the Nuclei of Bioassays, Ecophysiology and Biosynthesis of Natural Products (NuBBE), and the Universal Natural Products Database (UNPD), are collections of natural products. NuBBE is a virtual database of natural products and derivatives from the Brazilian biodiversity, whereas UNPD is a general resource of natural products created primarily for virtual screening and network pharmacology. Preferably redundancies between the datasets are removed.

The next two example sets, FDA-approved and Kyoto Encyclopedia of Genes and Genomes (KEGG) Drug, include molecules approved by regulatory agencies which possess acceptable risk versus benefit ratios. Although these molecules may still cause adverse drug reactions, they are referred to as non-toxic because of their relatively high therapeutic indices. FDA-approved drugs can be obtained from the DrugBank database, a widely used cheminformatics resource providing comprehensive information on known chemical reactions for drugs and their molecular targets. The KEGG-Drug resource contains drugs approved in Japan, United States, and Europe, annotated with the information on their targets, metabolizing enzymes, and molecular interactions. Again redundancies between the datasets can be removed.

Two example counter-datasets, TOXNET and the Toxin and Toxin Target Database (T3DB), contain compounds indicated to be toxic. The former resource maintained by the National Library of Medicine provides databases on toxicology, hazardous chemicals, environmental health, and toxic releases. Here, the system can use the Hazardous Substances Data Bank focusing on the toxicology of potentially hazardous chemicals. T3DB houses detailed toxicity data in terms of chemical properties, molecular and cellular interactions, and medical information, for a number of pollutants, pesticides, drugs, and food toxins. These data are extracted from multiple sources including other databases, government documents, books, and scientific literature. The non-redundant sets of TOXNET and T3DB contain 3035 and 1283 toxic compounds, respectively. As an independent set, one can employ the Traditional Chinese Medicine (TCM) Database@Taiwan, currently the largest and most comprehensive small molecule database on traditional Chinese medicine for virtual screening. As of this writing, TCM is based on information collected from Chinese medical texts and scientific publications for 453 different herbs, animal products, and minerals. From the original dataset, one can select molecules with a molecular weight in the range of 100-600 Da, and then remove redundancy at a TC of 0.8, producing a set of 5883 unique TCM compounds. Finally, the system can use the datasets to evaluate the prediction of specific toxicities. Compounds causing cancer in high dose tests can be, for example, obtained from the Carcinogenicity Potency (CP) database. These data are labeled based on series of experiments carried out on rodents considering different tissues of the subjects. A chemical is deemed toxic if it caused tumor growth in at least one tissue specific experiment. As of this writing, the CP set comprises 796 toxic and 605 non-toxic compounds. Likewise, as of this writing, the cardiotoxicity (CD) dataset contains 1571 molecules characterized with bioassay against human ether-a-go-go related gene (hERG) potassium channel. hERG channel blockade induces lethal arrhythmia causing a life-threatening symptom. The CD set includes 350 toxic compounds with an IC50 of <1 μM. The endocrine disruption (ED) dataset is prepared based on the bioassay data for androgen and estrogen receptors taken from the Tox21 Data Challenge. Endocrine disrupting chemicals interfere with the normal functions of endogenous hormones causing metabolic and reproductive disorders, the dysfunction of neuronal and immune systems, and cancer growth. As of this writing, the ED set contains 1317 toxic and 15,742 non-toxic compounds. The last specific dataset is focused on the acute oral toxicity (AO). Among 12,612 molecules with LD50 data provided by the SuperToxic database, 7392 compounds are labeled as toxic with a LD50 of <500 mg kg⁻¹. It is important to note that since LD50 is not indicative of non-lethal toxic effects, a chemical with a high LD50 may still cause adverse reactions at small doses.

The models of the system (whether identifying molecular fragments, potential synthetic molecules, filtering based on toxicity, etc.) rely on a common process, described here. While the specific data used to train those respective models may differ for a specific aspect of the system, the process remains the same. Input data to machine learning models are, for example, 1024-bit Daylight fingerprints constructed for dataset compounds with Open Babel (in other configurations, the size of the Daylight fingerprints may differ). The reference SAscore values can be computed with an exact approach that combines the fragment-based score representing the “historical synthetic knowledge” with the complexity-based score penalizing the presence of ring systems, such as spiro and fused rings, multiple stereo centers, and macrocycles. The DBN-based predictor of the SAscore can be trained and cross-validated against NuBBE, UNPD, FDA-approved, and DUD-E-active datasets. Cross-validation is used to evaluate the generalization of a trained model. In a k-fold cross-validation protocol, the system first divides the dataset into k different subsets and then the first subset is used as a validation set for a model trained on the remaining k−1 subsets. This procedure is repeated k times with the system employing different subsets as the validation set. Averaging the performance obtained for all k subsets yields the overall performance and estimates the validation error of the model.

Consider, as an example, the toxicity model, the Tox-score prediction can be conducted with a binary, ET-based classifier. The training and cross-validation can be carried out for the FDA-approved dataset used as positive (non-toxic) instances and the TOXNET dataset used as negative (toxic) instances. Subsequently, the toxicity predictor can be trained on the entire FDA-approved/TOXNET dataset and then independently tested against the KEGG-Drug (positive, non-toxic) and T3DB (negative, toxic) sets. In addition, the capability of the classifier to predict specific toxicities can be assessed against CP, CD, ED, and AO datasets. Similar to the SAscore predictor, a 5-fold cross-validation protocol can be employed to rigorously evaluate the performance of the toxicity classifier. Finally, both machine learning predictors of SAscore and Tox-score are applied to the TCM dataset.

The performance of a given module's prediction capability is assessed with several metrics derived from the confusion matrix, the accuracy (ACC), the sensitivity or true positive rate (TPR), and the fall-out or false positive rate (FPR):

$$\begin{matrix}ACC = \frac{T P + T N}{T P + F P + T N + F N} \\ TPR = \frac{T P}{T P + F N} \\ FPR = \frac{F P}{F P + T N}\end{matrix}$$

where TP is the number of true positives, i.e. non-toxic compounds classified as non-toxic (or other, similar metrics for other modules), and TN is the number of true negatives, (e.g., toxic compounds classified as toxic for the toxicity module). FP and FN are the numbers of over- and under-predicted molecules, respectively. In addition, the system assesses the overall quality of a binary classifier with the Matthews correlation coefficient (MCC) and the Receiver Operating Characteristic (ROC) analysis. The MCC is generally regarded as a well-balanced measure ranging from −1 (anti-correlation) to 1 (a perfect classifier) with values around 0 corresponding to a random guess:

$$M C C = \frac{TN \times TP - FP \times FN}{\sqrt{( T P + F P ) ( T P + F N ) ( T N + F P ) ( T N + F N )}}$$

where TP, TN, FP, and FN are defined above. The ROC analysis describes a trade-off between the FPR and the TPR for a classifier at varying decision threshold values. The MCC and ROC are important metrics to help select the best model considering the cost and the class distribution. The hyperparameters of the model, including the number of features resulting in the best split, the minimum number of samples required to split an internal node, and the minimum number of samples required to be at a leaf node, are tuned with a grid search method. The best set of hyperparameters maximizes both the MCC and ROC.

Finally, the performance of the regression classifier is evaluated with the mean squared error (MSE) and the Pearson correlation coefficient (PCC). The MSE is a risk function measuring the average of the squares of the errors:

$$M S E = \frac{1}{N} {\sum }_{i = 1}^{N} {( {\hat{y}}_{i} - {y}_{i} )}^{2}$$

where N is the total number of evaluation instances, and ŷᵢ and yᵢ are the predicted and actual values of i-th instance, respectively. Further, the PCC is often employed to assess the accuracy of point estimators by measuring the linear correlation between the predicted and actual values. Similar to the MCC, PCC ranges from −1 to 1, where −1 is a perfect anti-correlation, 1 is a perfect correlation, and 0 is the lack of any correlation. It is calculated as:

$$P C C = \frac{cov ( \hat{y} , y )}{{σ}_{\hat{y}} {σ}_{y}}$$

where cov(ŷ,y) is the covariance matrix of the predicted and actual values, and σ_{ŷ} and σ_{y} are the standard deviations of the predicted and actual values, respectively.

With respect to the toxicity score prediction, to quickly estimate the toxicity of large collections of low molecular weight organic compounds, the system can employ an ET classifier to compute the Tox-score ranging from 0 (a low probability to be toxic) to 1 (a high probability to be toxic). The primary dataset can consist of FDA-approved drugs, considered to be non-toxic, and potentially hazardous chemicals from the TOXNET database. Switching to an independent dataset causes the performance of machine learning classifiers to deteriorate on account of a fair amount of ambiguity in the training and testing sets. The disclosed system can estimate the toxicity of small organic compounds from their molecular fingerprints, and can provide discernible structural attributes of toxic and non-toxic substances. The output of the toxicity analysis can be a list of non-toxic possible candidates.

The goal after identifying the toxicity of various potential drugs is to create a model that can specifically correlate a pathogen, such as a bacterium or virus, and the specific drugs to which it is susceptible. This is accomplished by being able to accurately analyze which drugs are effective by understanding the protein-protein interactions (PPI) of the pathogen. For example, a drug's effects on a bacterium's internal proteins and the corresponding changes to the bacterium's PPI network can be crucial for understanding drug resistance. By using a mathematical representation of all physical contacts between proteins in a cell, the system can accurately predict whether a bacterium will be susceptible or resistant to a drug. The disclosed system is built as a graph convolutional network, where each protein is a node in the graph and each neighborhood of a node is the set of neighboring nodes in the protein structure. Each node has features computed from its amino sequence and structure, and edges have features describing interactions between residues. This network is a mathematical representation of all physical contacts between proteins in a cell.

The system disclosed herein has a similar architecture to most current graph convolutional networks. It's considered a convolutional network, as the filter parameters are shared over all nodes of a graph. To recognize specific signals or features of a graph, the network takes two things: 1) The graph structure as a series of nodes and edges, and 2) a feature description for every node, summarized as a feature matrix. The graph is built using the protein-protein interactions from the STRING dataset merged with the chemical-protein affinity data from the STITCH dataset. Protvec, a vector representation of protein sequences, is then added to each node as a feature. ProtVec uses an unsupervised data-driven distributed representation for biological sequences to represent the protein k-mer sequences as an n-dimensional vector. This allows for the protein to be defined by its vector in a context-aware manner, useful for neural network predictions or analysis. Mol2Vec is a vector representation for molecules, similar to the vector embedding created by ProtVec. The Mol2Vec is used to augment the system with specific molecular shapes of the antibiotics to further improve the network, and derive an understanding of specific antibiotic structure on the effects of antimicrobial resistance.

As additional data based on additional in-vitro/in-vivo testing is fed into the system, the convolutional network can be modified. This can result in a graph convolutional network which is constantly improving its capacity to make accurate predictions.

GCNs are used on this graph to create graph embeddings, contextual representations of each individual node in this graph. The embeddings, like fingerprints, capture the essential information of how the drug or protein interacts with in that specific bacteria mutation. By running these embeddings through a Multilayer Perceptron classifier, the system is able to predict the specific resistance of a bacteria species or strain to an antibiotic.

The preferred system disclosed herein for viral assessment can specifically correlate a virus and the specific drugs (or their combinations) that inhibit its different viral mechanisms. More specifically, the system can accurately analyze which drugs are effective by understanding the human-virus protein-protein interaction in the host cell. A drug's effect on viral mechanisms such as viral entry, RNA transcription, viral exit can be crucial for understanding the effectiveness of a therapy involving the drug. By using a mathematical representation of all physical contacts between proteins in the cell, the system can accurately predict whether a particular viral mechanism will possibly be inhibited a drug.

To recognize specific signals or features of the graph convolutional network, or a graph neural network, the network takes two things: 1) The graph structure as a series of nodes and edges, and 2) a feature description for every node, summarized as a feature matrix. where each protein is a node in the graph and each neighborhood of a node is assigned the set of neighboring nodes in the protein structure.

As an example, chemical nodes correspond to existing drugs in Drugbank, which contains data on 13491 approved and experimental drugs, or BindingDB, a large dataset of 1,908,553 binding data, for 7,605 protein targets and 846,857 small molecules. The edges can be target proteins. Peptide nodes correspond to Antiviral Peptides (AVP) in the AVPdb, a dataset of about 2683 AVPs as well as HIPdb, a dataset of 981 HIV peptides from varying sources tested on 35 different cell lines. A peptide node has edges to target mechanisms that are labeled by inhibition/IC50 weights.

The protein-protein and protein-virus node edges were derived from the following databases: HPIDB, hu.map, corum, and STRING. The Reactome dataset can be used for identifying specific protein mechanisms and labeling associated nodes.

A Graph Neural Network (GNN) technique called Node2Vec can be used to define a vector for every node within the graph. Graph-based AI techniques, such as Node2Vec, can be used to generate a “fingerprint” for drugs and AVPs that captures their properties and context within a mathematical representation of all cellular protein interactions. This mathematical representation can be created based on the previously described datasets. This allows for the protein to be defined by its vector in a context-aware manner, which can be useful for neural network predictions or analysis.

Target mechanisms also have edges to all proteins associated with them. The target mechanisms can include all possible viral target mechanisms from AVPdb along with other mechanisms such as glycolysis, ACE (Angiotensin-converting enzyme) receptors, and their associated proteins. The output of the graph convolutional network will be a vector that denotes the effectiveness weight for each drug for each target mechanism. A multicriteria optimization algorithm that operates on these vectors can be used to prioritize drugs and filter the top candidates (e.g., 10) that are most likely to succeed taking the toxicities (already included in the dataset) in consideration. These candidates can then be tested in a wetlab both in-vitro and in-vivo. In addition, scientists can test drug combinations and their effects on target mechanisms by merging chemical nodes. The training data for the graph convolutional network can include a subset of Drugbank that includes antivirals, some antibiotics, etc. The training dataset will also include peptide nodes.

The system can use two types of networks for the viral analysis: Siamese Networks (“SNets”) as well as Multilayer Perceptrons. The SNets make specific predictions based on a few AVPs especially important to the corona virus. SNets project fingerprints into multidimensional space and calculate distance between them within that dimensionality. The SNet produced very specific predictions based on a small number of optimal fingerprints. The Snet can provide separate predictions for the three mechanisms of antiviral action (entry, fusion, or replication), which afford a higher degree of specificity in drug selection. The SNet predictions are based on the similarity of a drug to the AVP fingerprints. The closer the prediction is to zero, the more similar a pair of fingerprints are and the more a drug resembles the dataset of AVPs. Predictions less than the optimal threshold indicates similar fingerprints, and therefore similar effect The Multilayered Perceptron (MLP) on the other hand, produces more general results based on at least 100 different AVPs. The result is represented by a ranking between 0 and 1, where 0 is the least effective and 1 is the most. A prediction above 0.2 is a good indication that the drug has an effect similar to the selected AVPs. The system can make four different predictions from the MLP: 1) Specific Virus: based on specific viral AVPs. 2) Entry: based on all AVPs (from other viruses as well) that target viral entry. 3) Fusion: based on all AVPs that target membrane fusion. 4) Replication: based on all AVPs that target replication. The advantage of the MLP is that it can summarize information from many AVPs and that there is very little randomness in the predictions. The disadvantage is that it cannot make specific predictions based on few AVPs. These seven networks together provide us a broad analysis by mechanism and viral specificity for a specific viral analysis.

A combination synthesis engine can be used to produce multi-drug therapies that can be synergetic. This allows us to predict which drugs combinations can be more effective than an individual monotherapy. In Vitro and In Vivo testing may be optimized based on the results of the network. Two exemplary engines which can be used within the combination synthesis engine include a DDI engine and a Synergy engine. The DDI (Drug-Drug Interaction) prediction network is based on an AI technique called multilayer perceptron, a feedforward Artificial Neural Network. By taking known DDI data from the DrugBank dataset as inputs, the system can predict a DDI between pairs of drugs. The data are based on DDI “templates” which include information on absorption, distribution, metabolism, excretion, and overall toxicity. Examples of effects resulting from drug-drug interactions ranged from increased cardiotoxic or hepatoxic activity, to increased anticoagulatory effect, or decreased absorption.

For example, for the COVID-19 disease state, several DDIs were determined to be positive outcomes which could assist in synergy between the drug pairs. An example of such a drug-drug interaction would be increased serum concentration of one drug (and increased effects) due to the second drug inhibiting an enzyme involved in the metabolism of the other drug. The predictions were weighed by outcome severity and relevance to the disease state before using the system to predict interactions from all permutations of the top drugs. If a DDI was predicted, outcome predictions were shown as two values: positive DDI score and negative DDI score. Since no DDIs were predicted for the imatinib mebendazole combination, both these scores were 0.

An example engine can be built based on datasets, such as DCDB (Drug Combinations Database), DrugCombDB, and Drugbank which was scraped for known PDDI (Potential Drug-Drug Interaction) and cytochrome p450 information. By using known FDA (Food and Drug Administration) approved synergistic combinations as positive examples and pairs of drugs that may lead to potential drug interactions as negative examples, the network can predict which combinations will lead to synergistic effects. In addition to synergistic efficacy, the system can also predict potentiating effects of one drug on another when used in combination. For example, the system's synergy analysis can use two main models: Bliss and Multi-dimensional synergy of combinations (MuSyC). The Bliss model is a reference model wherein the basic assumption is that the expected effect of a drug combination is two drugs acting independently, as if the two drugs were applied successively. Bliss reference effect:

y=1−product_all_drug(1−% Inhibition)*100.

For example, the system can use the Bliss model, which assumes that if two drugs do not exhibit any interactions the effect will be the same as the two drugs were acting independently (“additive”), receiving a Bliss score of 0. If they have reductive interactions, the score is less than zero, and if they have synergistic interactions the score would be greater than 0 (˜>10). A value just over zero indicates that the two drugs do not have any significant interactions.

Multi-dimensional synergy of combinations (MuSyC) is a model generated to additionally distinguish between potency and efficacy. The Bliss model does not distinguish between these and, as such, interpretations of the Bliss score could lead to misleading perception of the true combination interaction. The MuSyC model's beta score specifically indicates a drug combination's efficacy rather than any potentiating effect of Drug 1 upon Drug 2 or Drug 2 on Drug 1.

The overall process continues as follows: First, the system identifies known drugs and natural products that work on a single disease. Those drugs and natural products are run through molecular fragmentation to derive rigids and linkers that could be useful. Those rigids and linkers through the synthesis algorithms described to derive many derivative drugs from those initial rigids and linkers. This large list of drugs is filtered by toxicity and synthetic accessibility. The resulting list of drugs is then tested by for bacterial or viral specificity respectively. The system can then run these as combinations to find multi-drug therapies that may be more effective than a singular mono-therapy.

Having described the overall system, the disclosure turns to the figures.

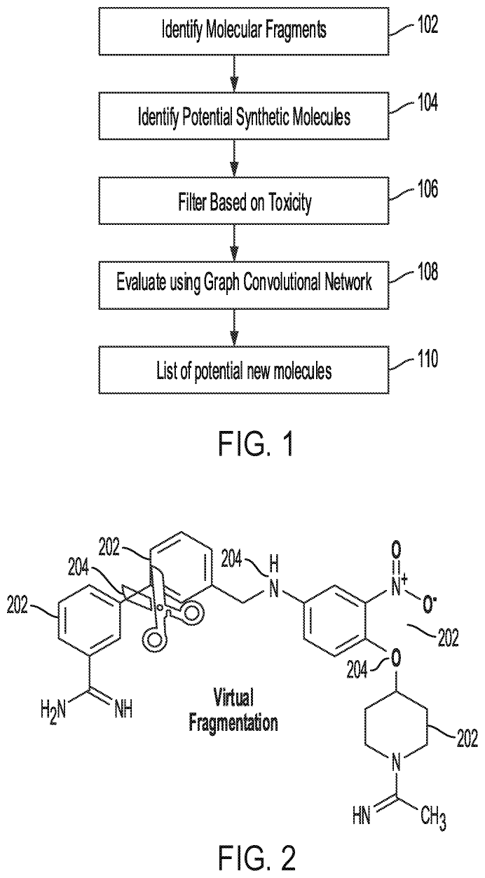

FIG. 1 illustrates a first example method of the invention. As illustrated, the system described herein first identifies molecular fragments 102 from molecules associated with a particular pathogen. For example, the system can identify multiple molecules which have previously been associated with similar pathogens and identify fragments within those molecules. The system can then identify potential synthetic molecules 104 based on known rules of chemistry. The resulting synthetic molecules identified by the system can be scored based on toxicity 106, and those molecules which have the highest scores can be processed using a graph convolutional network 108. The graph convolutional network allows for the molecules being tested to be flattened and compared to the pathogen, allowing for a determination of which molecules are likely to have desired effects on the pathogen. Those molecules which will have the desired effects can be ranked or otherwise provided as output from the system.

FIG. 2 illustrates a first example of fragmentation of molecules. In this figure, a given molecule being modeled by a computer system has various known sub-molecules 202 which are connected by one or more types of bonds 204. The system identifies the known sub-molecules 202 and fragments the overall molecule into those sub-molecules by cutting or removing the identified bonds 204.

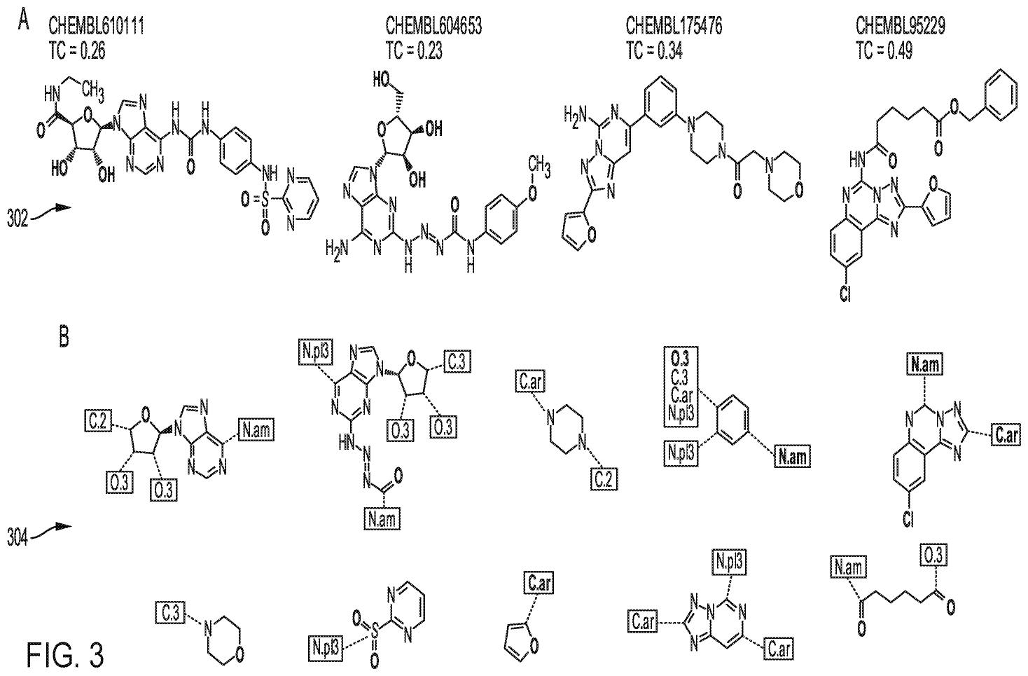

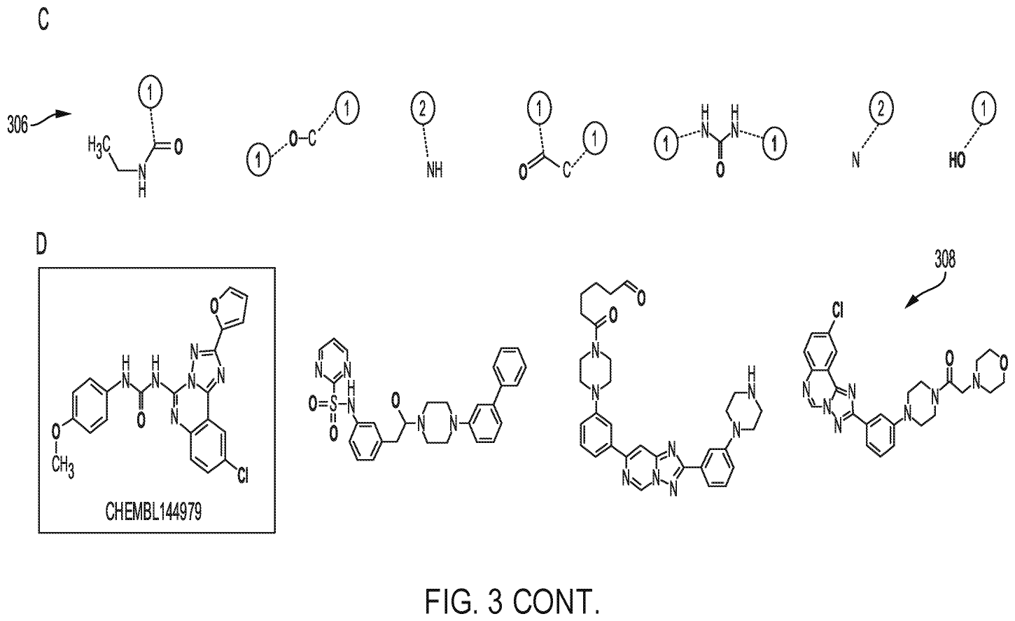

FIG. 3 illustrates a second example of fragmentation of molecules and synthesis of a bioactive. At step (A) 302, donor molecules with the chemical similarity to CHEMBL144979 are measured by the Tanimoto coefficient (TC). At step (B) 304, rigids are annotated with the list of atom types that can be attached at various positions. At step (C) 306 linkers are annotated with the number of the maximum allowed connections, completing the fragmentation process. Finally, at step (D) 308 new molecules are (virtually) synthesized using rigids and linkers. The first molecule shown in a box is a known bioactive of the adenosine receptor. Highlighted in different colors are essential building blocks to generate CHEMBL144979 that are extracted from donor molecules by the fragmentation and used in molecular synthesis. Further, the connectivity information inferred from donors that is required to correctly assemble CHEMBL144979 is highlighted in bold in (B) 304 and (C) 306.

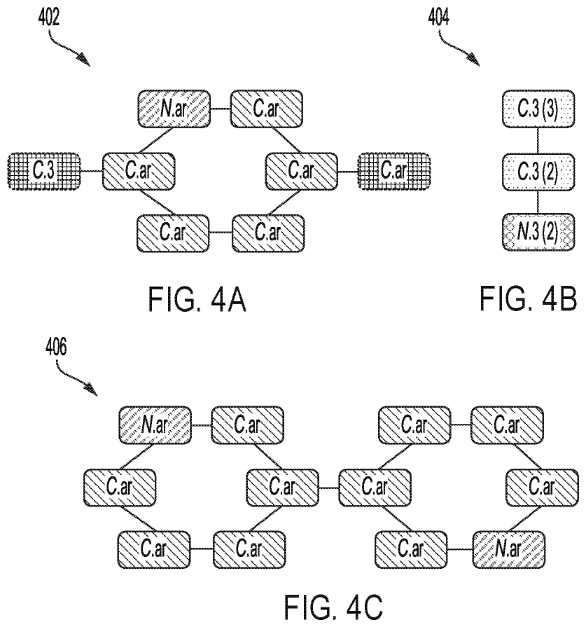

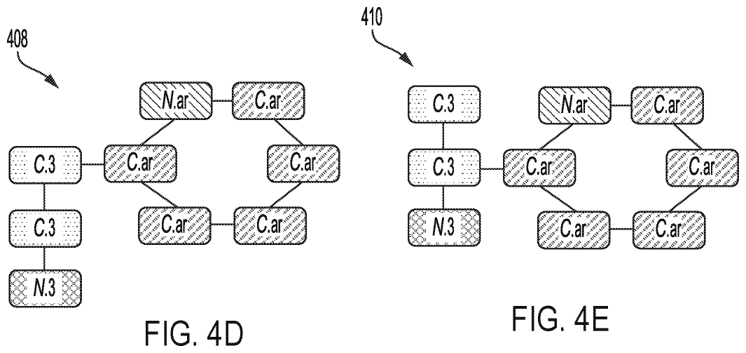

FIG. 4 illustrates a graph representation of rigids and linkers. (A) 402 illustrates the following sample molecular fragments: a rigid fragment, pyridine, with six constituent atoms in the bold outline and two possible connections to C.3 and C.ar in the dashed outline. (B) 404 illustrates a three-atom linking fragment containing C.3 carbon with up to 3 connections, C.3 carbon with up to 2 connections, and N.3 nitrogen with up to 2 connections. Examples of 2-molecules are shown in (C) 406, (D) 408, and (E) 410, with (C) 406 illustrating two identical rigids connected to each other. (D) 408 and (E) 410 respectively illustrate two possible ways of connecting the rigid and linker fragments shown in (A) 402 and (B) 404.

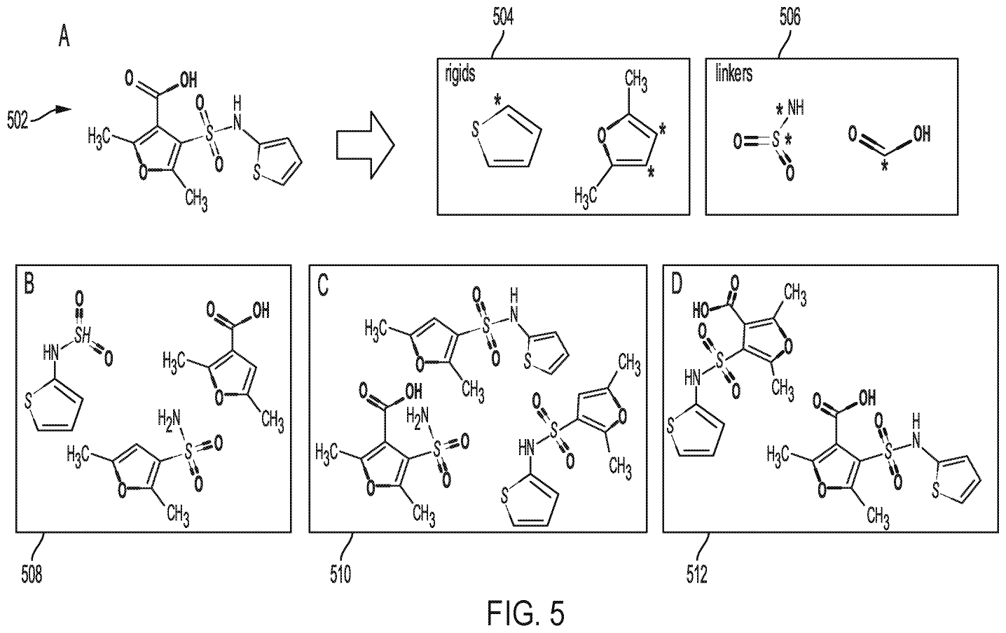

FIG. 5 illustrates an example of molecular synthesis. More specifically, FIG. 5 illustrates an example of the successful reconstruction of a molecule from its fragments. In (A) 502, the parent molecule is first decomposed into two rigids 504, thiophene (C₄H₄S) and 2,5-dimethylfuran [(CH₃)₂C₄H₂O], and two linkers 506, sulfonamide (SO₂N) and carboxylic acid [C(O)OH]. The rigids 504 and linkers 506 can then be used to construct (B) 508 2-molecules, (C) 510 3-molecules, and (D) 512 4-molecules including the parent compound.

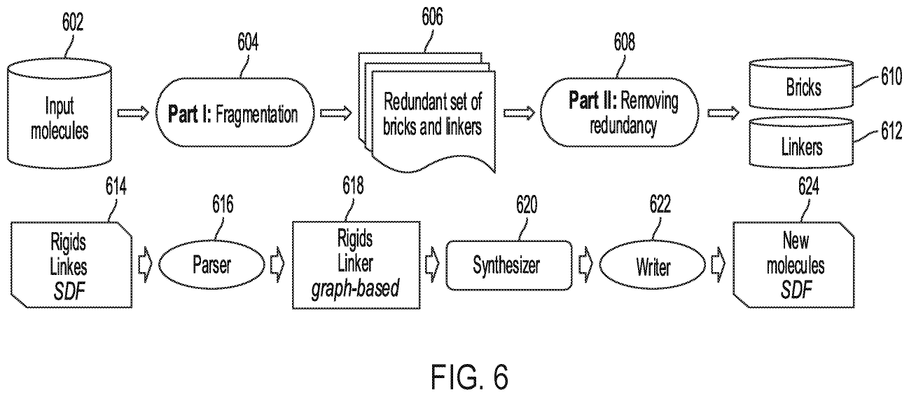

FIG. 6 illustrates an example of fragmentation and molecular synthesis. First, the input molecules 602 are received and fragmentation 604 occurs. This results in sets of rigids and linkers with redundancies 606, and the system removes those redundancies 608, resulting in a set of rigids 610 (also known as bricks) and a set of linkers 612. The rigids and linkers are stored in a Structure Data Format (SDF) file containing the 3D coordinates of all atoms and the corresponding atomic types as well as the connectivity information. The rigids and linkers 614 are then parsed 616, resulting in graph-based connections 618. The resulting graph is then used by a synthesizer 620 with known rules for chemical combinations, and the resulting synthesized chemicals are “generated” using a writer 622, with the result being new molecules 624 saved in the SDF format.

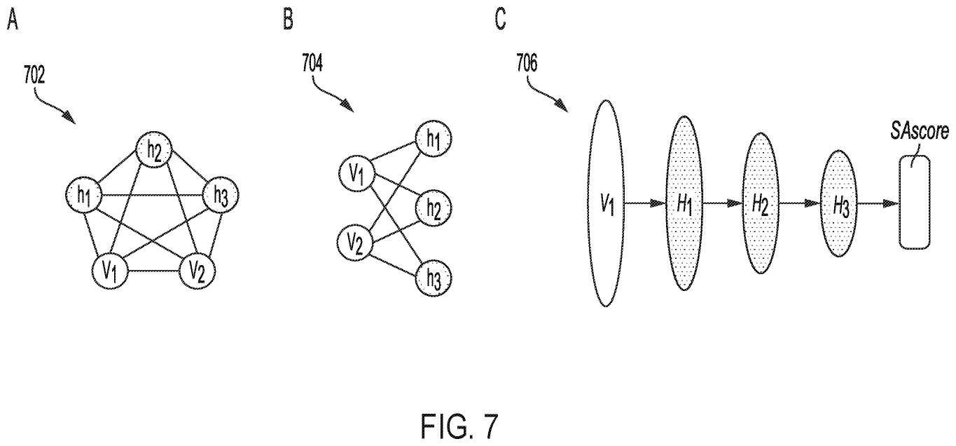

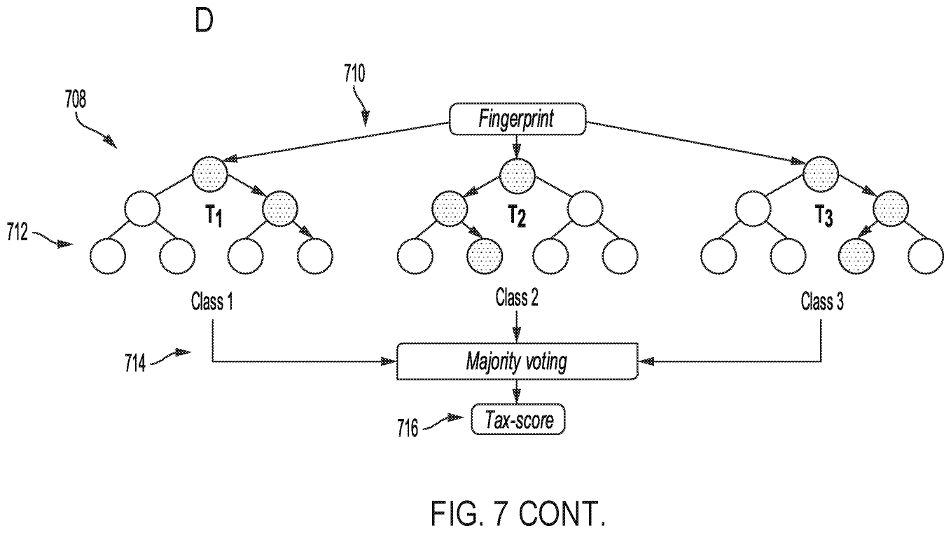

FIG. 7 illustrates an example of using machine learning to develop a toxicity score. (A) 702 illustrates a two-layered Boltzmann Machine with 3 hidden nodes h and 2 visible nodes v, with the nodes fully connected. (B) 704 illustrates a Restricted Boltzmann Machine (RBM) with the same nodes as in A. However, in this example the nodes belonging to the same layer are not connected. (C) 706 illustrates a Deep Belief Network with a visible layer V and 3 hidden layers H. Individual layers correspond to RBMs that are stacked against one another. (D) 708 illustrates a Random Forest with 3 trees T. For a given instance, each tree predicts a class based on a subset of the input set. The final class assignment is obtained by the majority voting of individual trees.

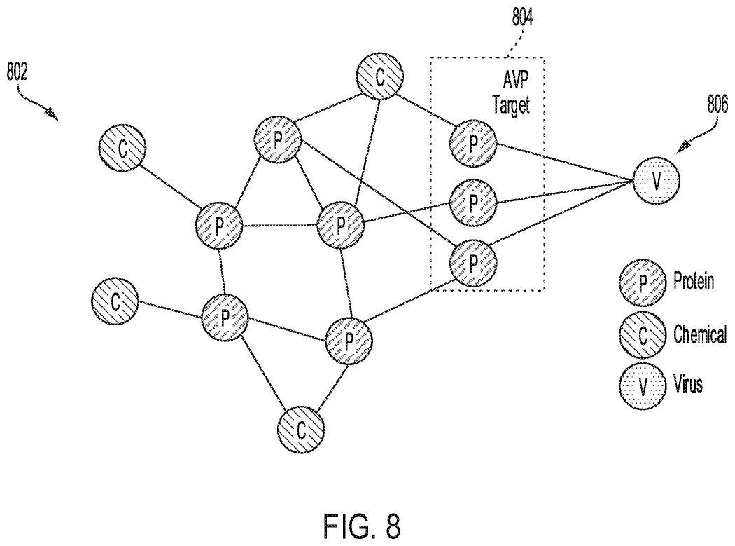

FIG. 8 illustrates an example of a graph convolutional network. In this example, which is directed to engaging with a virus 806, a molecule 802 under evaluation has a particular sequence of proteins 804, the “AVP target,” (Anti-Viral Peptite, “AVP”) which can interact with the virus in the intended manner. As the filtered molecules are evaluated using the convolutional network those molecules which do not contain the needed components (such as the AVP target 804) can be filtered or otherwise disregarded.

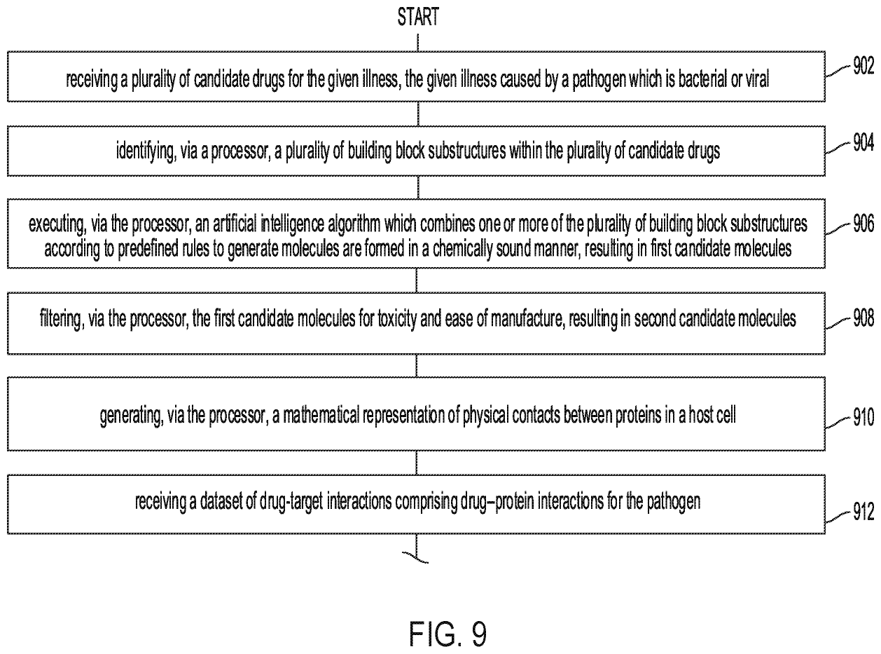

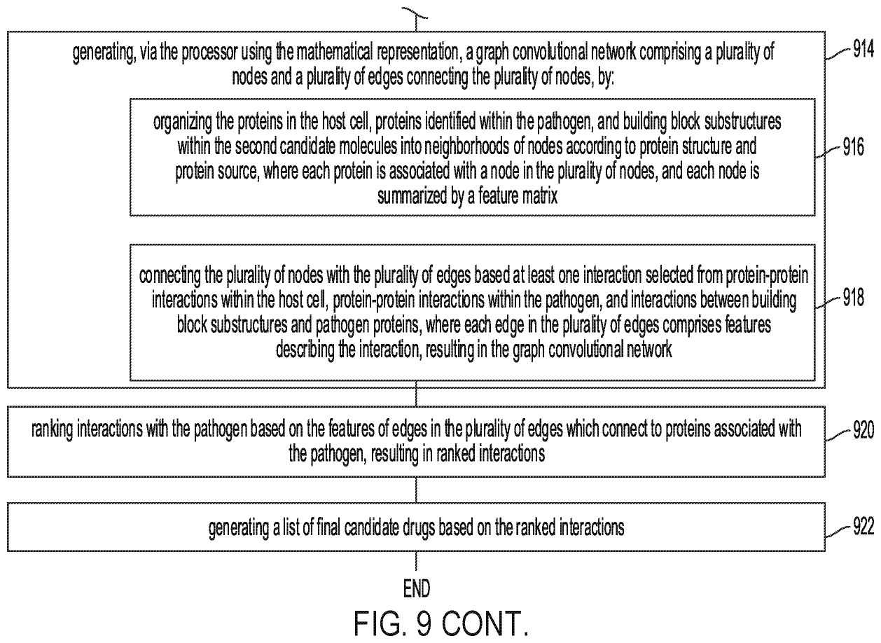

FIG. 9 illustrates an example method embodiment. In this example, the computer system executing the method receives a plurality of candidate drugs for the given illness, the given illness caused by a pathogen which is bacterial or viral (902). The system identifies, via a processor, a plurality of building block substructures within the plurality of candidate drugs (904) and executes, via the processor, an artificial intelligence algorithm which combines one or more of the plurality of building block substructures according to predefined rules to generate molecules are formed in a chemically sound manner, resulting in first candidate molecules (906). The system filters, via the processor, the first candidate molecules for toxicity and ease of manufacture, resulting in second candidate molecules (908).

The system generates, via the processor, a mathematical representation of physical contacts between proteins in a host cell (910) and receives a dataset of drug-target interactions comprising drug-protein interactions for the pathogen (912). The system then generates, via the processor using the mathematical representation, a graph convolutional network comprising a plurality of nodes and a plurality of edges connecting the plurality of nodes (914), by: organizing the proteins in the host cell, proteins identified within the pathogen, and building block substructures within the second candidate molecules into neighborhoods of nodes according to protein structure and protein source, where each protein is associated with a node in the plurality of nodes, and each node is summarized by a feature matrix (916); and connecting the plurality of nodes with the plurality of edges based at least in part on one interaction selected from protein-protein interactions within the host cell, protein-protein interactions within the pathogen, and interactions between building block substructures and pathogen proteins, where each edge in the plurality of edges comprises features describing the interaction, resulting in the graph convolutional network (918). The system then ranks interactions with the pathogen based on the features of edges in the plurality of edges which connect to proteins associated with the pathogen, resulting in ranked interactions (920) and generates a list of final candidate drugs based on the ranked interactions.

In some configurations, the illustrated method can further include: selecting, via the processor, a combination of at least two candidate drugs within the list of final candidate drugs predicted, by the processor, to have a synergistic therapeutic effect for the given illness. In such configurations, the synergistic therapeutic effect can be measured, at least in part, using a Bliss model.

In some configurations, the mathematical representation is generated via the processor using a Siamese Network.

In some configurations, the illustrated method can further include: virtually synthesizing, via the processor, each drug in the list of final candidate drugs.

In some configurations, at least one candidate drug in the final candidates is a combination of multiple drugs.

In some configurations, the illustrated method can further include ranking the final candidates drugs based on a difficulty of the virtual synthesizing process.

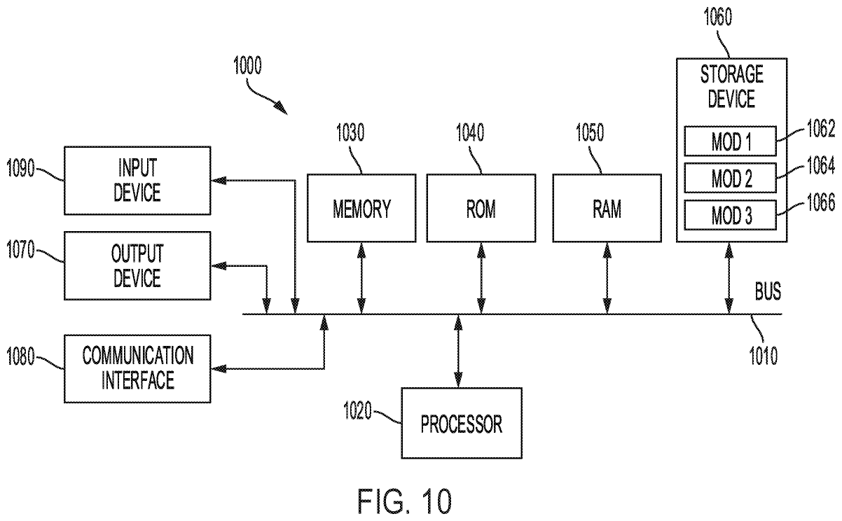

With reference to FIG. 10, an exemplary system includes a general-purpose computing device 1000, including a processing unit (CPU or processor) 1020 and a system bus 1010 that couples various system components including the system memory 1030 such as read-only memory (ROM) 1040 and random access memory (RAM) 1050 to the processor 1020. The system 1000 can include a cache of high-speed memory connected directly with, in close proximity to, or integrated as part of the processor 1020. The system 1000 copies data from the memory 1030 and/or the storage device 1060 to the cache for quick access by the processor 1020. In this way, the cache provides a performance boost that avoids processor 1020 delays while waiting for data. These and other modules can control or be configured to control the processor 1020 to perform various actions. Other system memory 1030 may be available for use as well. The memory 1030 can include multiple different types of memory with different performance characteristics. It can be appreciated that the disclosure may operate on a computing device 1000 with more than one processor 1020 or on a group or cluster of computing devices networked together to provide greater processing capability. The processor 1020 can include any general purpose processor and a hardware module or software module, such as module 1 1062, module 2 1064, and module 3 1066 stored in storage device 1060, configured to control the processor 1020 as well as a special-purpose processor where software instructions are incorporated into the actual processor design. The processor 1020 may essentially be a completely self-contained computing system, containing multiple cores or processors, a bus, memory controller, cache, etc. A multi-core processor may be symmetric or asymmetric.

The system bus 1010 may be any of several types of bus structures including a memory bus or memory controller, a peripheral bus, and a local bus using any of a variety of bus architectures. A basic input/output (BIOS) stored in ROM 1040 or the like, may provide the basic routine that helps to transfer information between elements within the computing device 1000, such as during start-up. The computing device 1000 further includes storage devices 1060 such as a hard disk drive, a magnetic disk drive, an optical disk drive, tape drive or the like. The storage device 1060 can include software modules 1062, 1064, 1066 for controlling the processor 1020. Other hardware or software modules are contemplated. The storage device 1060 is connected to the system bus 1010 by a drive interface. The drives and the associated computer-readable storage media provide nonvolatile storage of computer-readable instructions, data structures, program modules and other data for the computing device 1000. In one aspect, a hardware module that performs a particular function includes the software component stored in a tangible computer-readable storage medium in connection with the necessary hardware components, such as the processor 1020, bus 1010, display 1070, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 1000 is a small, handheld computing device, a desktop computer, or a computer server.

Although the exemplary embodiment described herein employs the hard disk 1060, other types of computer-readable media which can store data that are accessible by a computer, such as magnetic cassettes, flash memory cards, digital versatile disks, cartridges, random access memories (RAMs) 1050, and read-only memory (ROM) 1040, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 1000, an input device 1090 represents any number of input mechanisms, such as a microphone for speech, a touch-sensitive screen for gesture or graphical input, keyboard, mouse, motion input, speech and so forth. An output device 1070 can also be one or more of a number of output mechanisms known to those of skill in the art. In some instances, multimodal systems enable a user to provide multiple types of input to communicate with the computing device 1000. The communications interface 1080 generally governs and manages the user input and system output. There is no restriction on operating on any particular hardware arrangement and therefore the basic features here may easily be substituted for improved hardware or firmware arrangements as they are developed.

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure.

We claim:

1. A method for identifying therapeutics for a given illness, comprising:
obtaining, at a processor, a plurality of building block substructures contained within a plurality of candidate drugs for the given illness;
executing, via the processor, an artificial intelligence algorithm which combines one or more of the plurality of building block substructures according to predefined rules to generate molecules are formed in a chemically sound manner, resulting in first candidate molecules;
filtering, via the processor, the first candidate molecules for toxicity and ease of manufacture, resulting in second candidate molecules;
generating, via the processor, a mathematical representation of physical contacts between proteins in a host cell;
receiving a dataset of drug-target interactions comprising drug-protein interactions for the pathogen;
generating, via the processor using the mathematical representation, a graph convolutional network comprising a plurality of nodes and a plurality of edges connecting the plurality of nodes, by:
organizing the proteins in the host cell, proteins identified within the pathogen, and building block substructures within the second candidate molecules into neighborhoods of nodes according to protein structure and protein source, where each protein is associated with a node in the plurality of nodes, and each node is summarized by a feature matrix; and
connecting the plurality of nodes with the plurality of edges based at least in part on one interaction selected from protein-protein interactions within the host cell, protein-protein interactions within the pathogen, and interactions between building block substructures and pathogen proteins, where each edge in the plurality of edges comprises features describing the interaction, resulting in the graph convolutional network;
ranking interactions with the pathogen based on the features of edges in the plurality of edges which connect to proteins associated with the pathogen, resulting in ranked interactions; and
generating a list of final candidate drugs based on the ranked interactions.

2. The method of claim 1, further comprising:
selecting, via the processor, a combination of at least two candidate drugs within the list of final candidate drugs predicted, by the processor, to have a synergistic therapeutic effect for the given illness.

3. The method of claim 2, wherein the synergistic therapeutic effect is measured, at least in part, using a Bliss model.

4. The method of claim 1, wherein the mathematical representation is generated via the processor using a Siamese Network.

5. The method of claim 1, further comprising:
virtually synthesizing, via the processor, each drug in the final candidate drugs.

6. The method of claim 1, wherein the obtaining of the plurality of building block substructures comprises:
receiving the plurality of candidate drugs for the given illness, the given illness caused by a pathogen which is bacterial or viral; and
identifying, via the processor, the plurality of building block substructures within the plurality of candidate drugs.

7. A system comprising:
a processor; and
a non-transitory computer-readable storage medium having stored therein instructions which, when executed by the processor, cause the processor to perform operations comprising:
receiving a plurality of molecules known to have interactions with a pathogen;
fragmenting the plurality of molecules into a plurality of linkers and a plurality of rigids;
removing redundancies from the plurality of linkers and the plurality of rigids, resulting in fragments;
identifying a plurality of possible molecules formed from the fragments;
evaluating the plurality of possible molecules for toxicity, resulting in non-toxic possible candidates;
analyzing the non-toxic possible candidates using a convolutional neural network associated with the pathogen, resulting in final candidates; and
outputting the final candidates for in-vivo testing.

8. The system of claim 7, wherein the pathogen is either bacterial or viral.

9. The system of claim 7, the non-transitory computer-readable storage medium having stored therein at least one database of known chemical reactions.

10. The system of claim 7, wherein at least one candidate drug in the final candidates comprises a combination of multiple drugs

11. The system of claim 7, the non-transitory computer-readable storage medium having stored therein additional instructions which, when executed by the processor, cause the processor to perform operations comprising:
virtually synthesizing each drug in the final candidate.

12. The system of claim 11, the non-transitory computer-readable storage medium having stored therein additional instructions which, when executed by the processor, cause the processor to perform operations comprising:
ranking the final candidates based on a difficulty of the virtual synthesizing process.

13. The system of claim 11, the non-transitory computer-readable storage medium having stored therein additional instructions which, when executed by the processor, cause the processor to perform operations comprising:
selecting a combination of at least two candidate drugs within the final candidate drugs, wherein the combination is predicted, by the processor, to have a synergistic therapeutic effect for the pathogen.

14. The system of claim 13, wherein the synergistic therapeutic effect is measured, at least in part, using a Bliss model.

15. The system of claim 7, wherein the fragments are in a mathematical representation generated via the processor using a Siamese Network.

16. The system of claim 7, wherein the plurality of linkers and the plurality of rigids are stored in a Structure Data Format file within the non-transitory computer-readable storage medium.

17. The system of claim 7, wherein the evaluating of the plurality of possible molecules for toxicity is performed by the processor executing at least one of an Extremely Randomized Trees algorithm and an Extra Trees (ET) algorithm.

18. A non-transitory computer-readable storage medium having stored therein instructions which, when executed by a processor, cause the processor to perform operations comprising:
receiving a plurality of molecules known to have interactions with a pathogen;
fragmenting the plurality of molecules into a plurality of linkers and a plurality of rigids;
removing redundancies from the plurality of linkers and the plurality of rigids, resulting in fragments;
identifying a plurality of possible molecules formed from the fragments;
evaluating the plurality of possible molecules for toxicity, resulting in non-toxic possible candidates;
analyzing the non-toxic possible candidates using a convolutional neural network associated with the pathogen, resulting in final candidates; and
outputting the final candidates for in-vivo testing.

19. The non-transitory computer-readable storage medium of claim 18, wherein the pathogen is bacterial.

20. The non-transitory computer-readable storage medium of claim 18, wherein the pathogen is viral.
